(19) United States
(12) Patent Application Publication
(10) Pub. No.: US 2022/0362318 A1
(43) Pub. Date: Nov. 17, 2022

(54) METHOD FOR TREATMENT OF PSORIASIS

(71) Applicant: Shaochi HSIN, New Taipei City (TW)

(72) Inventor: Shaochi HSIN, New Taipei City (TW)

(21) Appl. No.: 17/662,989

(22) Filed: May 11, 2022

Related U.S. Application Data
(60) Provisional application No. 63/187,092, filed on May 11, 2021.

(51) Int. Cl. A61K 36/074; A61K 9/00; A61K 38/00; A61K 47/38; A61K 47/10; A61P 29/00

(52) U.S. Cl. CPC A61K 36/074; A61K 9/006; A61K 38/00; A61K 47/38; A61K 47/10; A61P 29/00

(57) ABSTRACT

The present disclosure provides a method for treating or alleviating psoriasis, and/or promoting or accelerating the healing of psoriasis, comprising administering a pharmaceutical composition comprising an effective amount of an immunomodulatory protein derived from Ganoderma.

### PRIORITY INFORMATION

This application claims priority to and benefit of U.S. Provisional Application No. 63/187,092, filed May 11, 2021, the contents of which are incorporated herein.

### FIELD OF THE INVENTION

The present disclosure relates to a method for treatment of psoriasis, and particularly, a method utilizing an immunomodulatory protein derived from Ganoderma.

### BACKGROUND OF THE INVENTION

Psoriasis is a long-lasting, noncontagious autoimmune disease characterized by raised areas of abnormal skin, most commonly on the knees, elbows, trunk and scalp. These areas are typically red or purple on some people with darker, dry, itchy, and scaly skin. It tends to attack periodically, flaring up for a few weeks or months, then subsiding or going into remission. Common triggers for psoriasis include stress, alcohol, injury, and medication.

Psoriasis varies in severity from small, localized patches to complete body coverage, with possible mechanisms complicated and systemic. It remains a challenge to manage psoriasis in a safe and economical manner.

Systemic therapy such as methotrexate, or biologics such as etanercept, adalimumab, infliximab, etc., are used for treatment when the skin involvement is extensive, e.g., ten percent of the body surface area or more. A large number of those suffering from psoriasis have less extensive effect, and topical medications are considered a safer and more prudent alternative in most of these cases. Among topical therapies are anti-inflammatory corticosteroids, particularly super potent varieties such as halobetasol propionate, vitamin D derivatives, such as calcipotriene, a retinoid known as tazarotene, and coal tar. While each of the topical therapies provides a particular degree of effectiveness, limitations are present in the degree to which they can improve psoriatic plaques, or in adverse effects generated.

### SUMMARY OF THE INVENTION

The present disclosure surprisingly found that an immunomodulatory protein derived from Ganoderma or a recombinant thereof provides advantageous efficacy in treating or alleviating psoriasis, and/or promoting or accelerating the healing of psoriasis. Accordingly, the present disclosure provides a method for treatment of psoriasis with an immunomodulatory protein derived from Ganoderma.

In one aspect, the present disclosure provides a method for treating or alleviating psoriasis, and/or promoting or accelerating the healing of psoriasis, including administering a pharmaceutical composition to a subject in need thereof. The pharmaceutical composition comprises an effective amount of an immunomodulatory protein derived from Ganoderma, or a recombinant or fragment thereof.

In some embodiments of the disclosure, the immunomodulatory protein comprises an amino acid sequence of SEQ ID NO: 3 or 4. In some embodiments of the disclosure, the fragment of immunomodulatory protein comprises an amino acid sequence selected from the group consisting of SEQ ID NOs: 1 to 2.

The sequences of SEQ ID NOs: 1 to 4 are as follows.

(SEQ ID NO: 1)
LAWNVK
(SEQ ID NO: 2)
DLGVRPSYAV
(SEQ ID NO: 3)
MSDTALIFTLAWNVKQLAFDYTPNWGRGRPSSFIDTVTFPTVLTDKAYTY
RVVVSGKDLGVRPSYAVESDGSQKINFLEYNSGYGIADTNTIQVYVIDPD
TGNNFIVAQWN
(SEQ ID NO: 4)
EAEAEFMSDTALIFTLAWNVKQLAFDYTPNWGRGRPSSFIDTVTFPTVLT
DKAYTYRVVVSGKDLGVRPSYAVESDGSQKINFLEYNSGYGIADTNTIQV
YVIDPDTGNNFIVAQWNYLEQKLISEEDLNSAVDHHHHHH

In some embodiments of the disclosure, the pharmaceutical composition further comprises a gel-forming agent.

In some embodiments of the disclosure, the gel forming agent is in an amount from about 0.1% (w/w) to about 2% (w/w), about 0.15% (w/w) to about 1.95% (w/w), about 0.2% (w/w) to about 1.9% (w/w), about 0.25% (w/w) to about 1.85% (w/w), about 0.3% (w/w) to about 1.8% (w/w), about 0.35% (w/w) to about 1.75% (w/w), about 0.4% (w/w) to about 1.7% (w/w), about 0.45% (w/w) to about 1.65% (w/w), about 0.5% (w/w) to about 1.6% (w/w), about 0.55% (w/w) to about 1.55% (w/w), about 0.6% (w/w) to about 1.5% (w/w), about 0.65% (w/w) to about 1.45% (w/w), about 0.7% (w/w) to about 1.4% (w/w), about 0.75% (w/w) to about 1.35% (w/w), about 0.8% (w/w) to about 1.3% (w/w), about 0.85% (w/w) to about 1.25% (w/w), about 0.9% (w/w) to about 1.2% (w/w), about 0.95% (w/w) to about 1.15% (w/w), about 1.0% (w/w) to about 1.1% (w/w) in the pharmaceutical composition. In some embodiments, the amount of the gel-forming agent ranges from about 0.5% (w/w) to about 2.0% (w/w), about 0.5 (w/w) to about 1.5% (w/w), about 0.5 (w/w) to about 1.2% (w/w), about 0.5 (w/w) to about 1.0% (w/w), about 0.1 (w/w) to about 1.5% (w/w), about 0.1 (w/w) to about 1.0% (w/w), about 0.1 (w/w) to about 0.5% (w/w), about 1.0% (w/w) to about 2.0% (w/w), or about 1.5% (w/w) to about 2% (w/w).

In some embodiments of the disclosure, the immunomodulatory protein is in an amount from about 0.0001% (w/w) to about 0.05% (w/w), about 0.0001% (w/w) to about 0.05% (w/w), about 0.0005% (w/w) to about 0.045% (w/w), about 0.001% (w/w) to about 0.04% (w/w), about 0.005% (w/w) to about 0.035% (w/w), about 0.001% (w/w) to about 0.03% (w/w), about 0.005% (w/w) to about 0.025% (w/w), about 0.01% (w/w) to about 0.02% (w/w), about 0.0001% (w/w) to about 0.05% (w/w), about 0.0001% (w/w) to about 0.05% (w/w), about 0.0001% (w/w) to about 0.05% (w/w), about 0.0001% (w/w) to about 0.05% (w/w), about 0.0001% (w/w) to about 0.05% (w/w), about 0.0001% (w/w) to about 0.05% (w/w), about 0.0001% (w/w) to about 0.05% (w/w), about 0.0001% (w/w) to about 0.05% (w/w), in the pharmaceutical composition. In some embodiments, the amount of the immunomodulatory protein ranges from about 0.0001% (w/w) to about 0.03% (w/w), about 0.0001% (w/w) to about 0.01% (w/w), about 0.0001% (w/w) to about 0.03% (w/w), about 0.0001% (w/w) to about 0.01% (w/w), about 0.0001% (w/w) to about 0.005% (w/w), about 0.0001% (w/w) to about 0.003% (w/w), about 0.0001% (w/w) to about 0.001% (w/w) or about 0.0001% (w/w) to about 0.0005% (w/w).

Examples of gel-forming agents include, but are not limited to, polyethylene glycol (PEG)-diacrylate, PEG-acrylate, PEG-thiol, PEG-azide, PEG-alkyne, chitosan, hyaluronic acid, collagen, fibrin, acacia, alginic acid, natto gum, aloe vera, bentonite, carbomers, carboxymethyl cellulose, ethylcellulose, gelatin, elastin, hydroxypolyamide, hydroxyethyl cellulose, hydroxypropyl cellulose, hydroxypropyl methylcellulose, magnesium aluminum silicate, methylcellulose, poloxamers, polyvinyl alcohol, sodium alginate, tragacanth, xanthan gum, gelatin, carboxyvinyl polymers, starch, water-swellable hydrocolloids, carragenans, hyaluronates, agarose, alginates, acrylates and ammonium acryloyldimethyltaurate/vinyl pyrrolidone (VP) copolymer.

In some embodiments, the gel-forming agent is xanthan gum, methylcellulose, or ammonium acryloyldimethyltaurate/VP copolymer.

In one embodiment, the pharmaceutical composition has a pH ranging from about 5.5 to about 7.5. In some embodiments, the pH ranges from about 6.0 to about 7.5, about 6.5 to about 7.5, about 5.5 to about 7.0, about 6.0 to about 7.0, about 6.5 to about 7.0, about 5.5 to about 6.5, or about 6.0 to about 6.5.

In one embodiment, the pharmaceutical composition has a viscosity ranging from about 0.05 Pa·s to about 200 Pa·s. In some embodiments, the viscosity ranges from about 0.1 Pa·s to about 200 Pa·s, about 0.5 Pa·s to about 200 Pa·s, about 1.0 Pa·s to about 200 Pa·s, about 5 Pa·s to about 200 Pa·s, about 10.0 Pa·s to about 200 Pa·s, about 20.0 Pa·s to about 200 Pa·s, about 40.0 Pa·s to about 200 Pa·s, about 60.0 Pa·s to about 200 Pa·s, about 80.0 Pa·s to about 200 Pa·s, about 100.0 Pa·s to about 200 Pa·s, about 120 Pa·s to about 200 Pa·s, about 140 Pa·s to about 200 Pa·s, about 160 Pa·s to about 200 Pa·s, about 0.05 Pa·s to about 160 Pa·s, 0.05 Pa·s to about 140 Pa·s, 0.05 Pa·s to about 120 Pa·s, 0.05 Pa·s to about 100 Pa·s, 0.05 Pa·s to about 80 Pa·s, 0.05 Pa·s to about 60 Pa·s, 0.05 Pa·s to about 40 Pa·s, 0.05 Pa·s to about 20 Pa·s, 0.05 Pa·s to about 10 Pa·s, 0.05 Pa·s to about 5.0 Pa·s, 0.05 Pa·s to about 3.0 Pa·s or 0.05 Pa·s to about 1.0 Pa·s.

In some embodiments, the pharmaceutical composition is topically administered on an area of psoriasis.

In one embodiment, the pharmaceutical composition is topically administered on an area of psoriasis, and the effective amount of the immunomodulatory protein ranges from about 1 mcg/cm² to about 100 mcg/cm², about 1 mcg/cm² to about 80 mcg/cm², about 1 mcg/cm² to about 60 mcg/cm², about 1 mcg/cm² to about 40 mcg/cm², about 1 mcg/cm² to about 20 mcg/cm², about 1 mcg/cm² to about 10 mcg/cm², about 1 mcg/cm² to about 5 mcg/cm², about 5 mcg/cm² to about 100 mcg/cm², about 10 mcg/cm² to about 100 mcg/cm², about 20 mcg/cm² to about 100 mcg/cm², about 40 mcg/cm² to about 100 mcg/cm², about 60 mcg/cm² to about 100 mcg/cm² or about 80 mcg/cm² to about 100 mcg/cm² of the area of psoriasis.

In some embodiments of the disclosure, the pharmaceutical composition is orally administered to the subject.

In some embodiments of the disclosure, the pharmaceutical composition is orally administered to the subject, the effective amount of the immunomodulatory protein ranges from about 0.01 mg/kg to about 5 mg/kg. In some embodiments, the effective amount ranges from about 0.01 mg/kg to about 4 mg/kg, about 0.01 mg/kg to about 4.5 mg/kg, about 0.01 mg/kg to about 4 mg/kg, about 0.01 mg/kg to about 3.5 mg/kg, about 0.01 mg/kg to about 3 mg/kg, about 0.01 mg/kg to about 2.5 mg/kg, about 0.01 mg/kg to about 2 mg/kg, about 0.01 mg/kg to about 1.5 mg/kg, about 0.01 mg/kg to about 1 mg/kg, about 0.01 mg/kg to about 0.5 mg/kg, about 0.05 mg/kg to about 5 mg/kg, about 0.05 mg/kg to about 4.5 mg/kg, about 0.05 mg/kg to about 4.5 mg/kg, about 0.05 mg/kg to about 4 mg/kg, about 0.05 mg/kg to about 3.5 mg/kg, about 0.05 mg/kg to about 3 mg/kg, about 0.05 mg/kg to about 2.5 mg/kg, about 0.05 mg/kg to about 2 mg/kg, about 0.05 mg/kg to about 1.5 mg/kg, about 0.05 mg/kg to about 1 mg/kg, about 0.05 mg/kg to about 0.5 mg/kg, about 0.1 mg/kg to about 5 mg/kg, about 0.1 mg/kg to about 4.5 mg/kg, about 0.1 mg/kg to about 4 mg/kg, about 0.1 mg/kg to about 3.5 mg/kg, about 0.1 mg/kg to about 3 mg/kg, about 0.1 mg/kg to about 2.5 mg/kg, about 0.1 mg/kg to about 2 mg/kg, about 0.1 mg/kg to about 1.5 mg/kg, about 0.1 mg/kg to about 1 mg/kg, about 0.1 mg/kg to about 0.5 mg/kg, about 0.5 mg/kg to about 5 mg/kg, about 1 mg/kg to about 5 mg/kg, about 1.5 mg/kg to about 5 mg/kg, about 2 mg/kg to about 5 mg/kg, about 2.5 mg/kg to about 5 mg/kg, about 3 mg/kg to about 5 mg/kg, about 3.5 mg/kg to about 5 mg/kg or about 4 mg/kg to about 5 mg/kg.

In one embodiment, the method further comprises administering one or more additional therapeutic agents to the subject. In some embodiments, the therapeutic agent includes topical medication (e.g., corticosteroids, vitamin D analogues, retinoids, calcineurin inhibitors, salicylic acid, coal tar, anthralin), light (e.g., sunlight, UVB broadband, UVB narrowband, psoralen plus ultraviolet A (PUVA), excimer laser) or oral or intravenous application (e.g., steroids, retinoids, methotrexate, cyclosporine, etanercept (Enbrel), infliximab (Remicade), adalimumab (Humira), ustekinumab (Stelara), secukinumab (Cosentyx), ixekizumab (Taltz), thioguanine (Tabloid), hydroxyurea (Droxia, Hydrea), Apremilast (Otezla)).

In some embodiments, the psoriasis is plaque psoriasis, pustular psoriasis, inverse psoriasis, napkin psoriasis, guttate psoriasis, oral psoriasis, erythrodermic psoriasis, seborrheic-like psoriasis, nail psoriasis, or psoriatic arthritis.

In some embodiments, the psoriasis is familial psoriasis.

### BRIEF DESCRIPTION OF THE DRAWING

FIGS. 1A and 1B show the effects of the treatment of psoriasis with the hydrogel of the present disclosure in patent 1. FIG. 1A: before treatment; FIG. 1B: after treatment for 2 months.

FIGS. 2A and 2B show the effects of the treatment of psoriasis with the capsule of the present disclosure in patent 1. FIG. 2A: before treatment; FIG. 2B: after treatment for 4 months.

FIGS. 3A to 3C show the effects of the treatment of psoriasis with the capsule of the present disclosure in patent 2. FIG. 3A: treatment on week 3; FIG. 3B: treatment on week 12 (skin); FIG. 3C: treatment on week 12 (nail).

FIGS. 4A to 4C show the effects of the treatment of psoriasis with the capsule of the present disclosure in patent 3. FIG. 4A: before treatment; FIG. 4B: treatment on week 2; FIG. 4C: treatment on week 4.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned are incorporated herein for reference.

In this application, the use of the singular includes the plural, the article “a” or “an” mean “at least one,” and the use of “or” means “and/or,” unless specifically stated otherwise.

The term “topical” refers to administration or delivery of a compound by application of the compound to a surface of a body part.

As used herein, “promote” or “increase,” or “promoting” or “increasing” are used interchangeably. These terms refer to the increase in a measured parameter in a treated cell, tissue or subject in comparison with an untreated cell, tissue or subject. A comparison can also be made of the same cell or tissue or subject before and after treatment. In some embodiments, the increase in the treated cell, tissue or subject is at least about 10%, 20%, 30%, 40%, 50%, 60%, 70%, 80%, 90%, 1-fold, 2-fold, 3-fold, 4-fold or more in comparison with an untreated cell, tissue or subject.

As used herein, the term “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “treating” or “treatment” (and grammatical variations thereof such as “treat”) refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state and remission or improved prognosis.

The term “effective amount” of an active ingredient as provided herein means a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

As used herein, the term “topical formulation” (or “topical composition”) is used to refer to a pharmaceutical preparation intended for topical or local application to an afflicted region of a subject in need thereof.

As used herein, the term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The present disclosure provides a method for treating or alleviating psoriasis, and/or promoting or accelerating the healing of psoriasis, the method comprising administrating a pharmaceutical composition to a subject in need thereof, wherein the pharmaceutical composition comprises an effective amount of an immunomodulatory protein derived from Ganoderma, or a recombinant or fragment thereof.

In one embodiment, the immunomodulatory protein or a recombinant thereof or a fragment thereof is derived from Ganoderma lucidum, Ganoderma lucidum, Ganoderma tsugae, Ganoderma microsporum or Ganoderma sinensis. In some embodiments, the immunomodulatory protein is an immunomodulatory protein or a recombinant thereof which comprises comprises an amino acid sequence of SEQ ID NO: 3 or 4. In some embodiments, the fragment of immunomodulatory protein comprises an amino acid sequence selected from the group consisting of SEQ ID NOs: 1 to 2. The preparation of the immunomodulatory protein or a recombinant thereof has been described in U.S. Pat. No. 7,601,808.

In some embodiments, treating or alleviating psoriasis, and/or promoting or accelerating the healing of psoriasis may include reducing inflammation and scales, slowing growth of skin cells, and/or removing plaques.

The immunomodulatory protein or a recombination thereof or a fragment thereof or pharmaceutical composition as disclosed herein can be administered to a subject either alone or in pharmaceutical compositions where it is mixed with suitable carriers and excipients. The immunomodulatory protein, or a recombination thereof or a fragment thereof or pharmaceutical composition as disclosed herein can be administered parenterally, such as by intravenous injection or infusion, intraperitoneal injection, subcutaneous injection, or intramuscular injection. The immunomodulatory protein or a recombination thereof or a fragment thereof or pharmaceutical composition as disclosed herein can be administered orally or rectally through appropriate formulation with carriers and excipients to form tablets, pills, capsules, liquids, gels, syrups, slurries, suspensions and the like. The immunomodulatory protein or a recombination thereof or a fragment thereof or pharmaceutical composition as disclosed herein can be administered topically, such as by skin patch. The immunomodulatory protein or a recombination thereof or a fragment thereof or pharmaceutical composition as disclosed herein can be formulated into topical creams, skin or mucosal patch, or liquids or gels suitable to topical application to skin or mucosal membrane surfaces. The immunomodulatory protein or a recombination thereof or a fragment thereof or pharmaceutical composition as disclosed herein can be administered by inhaler to the respiratory tract for local or systemic treatment of psoriasis.

The dosage of the immunomodulatory protein or a recombination thereof or a fragment thereof or pharmaceutical composition can be determined by those skilled in the art on the basis of the disclosure herein. The medicament will contain an effective dosage (depending upon the route of administration and pharmacokinetics of the active agent) of suitable pharmaceutical carriers and excipients suitable for the particular route of administration of the formulation (i.e., oral, parenteral, topical or by inhalation). The immunomodulatory protein or a recombination thereof or a fragment thereof is mixed into the pharmaceutical composition by means of mixing, dissolving, granulating, dragee-making, emulsifying, encapsulating, entrapping or lyophilizing processes. The pharmaceutical compositions for parenteral administration include aqueous solutions of the inventive polypeptide in water-soluble form. Additionally, suspensions of the inventive polypeptide may be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may optionally contain stabilizers or agents to increase the solubility of the complex or combination to allow for more concentrated solutions.

In some embodiments, the pharmaceutical composition is intended to be topically applied in various ways which will be further described. For example, the pharmaceutical composition can be provided as a hydrogel. In some embodiments of the disclosure, the pharmaceutical composition further comprises a gel-forming agent.

In some embodiments of the disclosure, the gel forming agent is in an amount from about 0.1% (w/w) to about 2% (w/w) in the pharmaceutical composition.

In some embodiments of the disclosure, the immunomodulatory protein is in an amount from about 0.0001% (w/w) to about 0.05% (w/w) in the pharmaceutical composition.

The pharmaceutical composition of the present disclosure also comprises a gel-forming agent to form a topical gel product with a viscosity ranging from about 0.05 Pa·s to about 200 Pa·s.

The pharmaceutical composition of the present disclosure is formulated as having a pH from 5.5 to 7.5. In one embodiment, the pH of the aqueous medium can be adjusted by means of low concentrations of suitable biocompatible buffering ingredients, non-limiting examples being tromethamine, sodium carbonate and bicarbonate, as well as sodium dihydrogen phosphate and disodium hydrogen phosphate.

The pharmaceutical composition of the present disclosure may also include further additives such as solvents, gel forming/polymeric agents, viscosity increasing agents, emulsifiers, antioxidants, preservatives, pH adjusting agents, propellants and combinations of the foregoing.

In some embodiments of the disclosure, the pharmaceutical composition is orally administered to the subject. The carrier is used as a carrier and/or diluent and/or adjuvant, or vehicle for delivery of a therapeutic agent to a subject or added to a formulation to improve its handling or storage properties or to permit or facilitate formation of a dose unit of the composition into a discrete article such as a capsule or tablet suitable for oral administration. Suitable carriers are well known to persons of ordinary skill in the art of manufacturing pharmaceutical formulations or food products. Carriers can include, by way of illustration and not limitation, buffers, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable carriers include citrate buffer, phosphate buffer, acetate buffer, bicarbonate buffer, stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials (such as cellulose esters of alkanoic acids and cellulose alkyl esters), low melting wax cocoa butter, amino acids, urea, alcohols, ascorbic acid, phospholipids, proteins (for example, serum albumin), ethylenediamine tetraacetic acid (EDTA), dimethyl sulfoxide (DMSO), sodium chloride or other salts, liposomes, mannitol, sorbitol, glycerol or powder, polymers (such as polyvinyl-pyrrolidone, polyvinyl alcohol, and polyethylene glycols), and other pharmaceutically acceptable materials. The carrier should not destroy the pharmacological activity of the therapeutic agent and should be non-toxic when administered in doses sufficient to deliver a therapeutic amount of the agent.

In some embodiments of the disclosure, the pharmaceutical composition is orally administered to the subject, the effective amount of the immunomodulatory protein ranges from about 0.01 mg/kg to about 5 mg/kg. In some further embodiments of the disclosure, the effective amount of the immunomodulatory protein ranges from about 0.1 mg/kg to about 3 mg/kg.

In some embodiments, the method further comprises administering one or more therapeutic agents to the subject. The therapeutic agent may be any known medication for treatment of psoriasis, such as topical medication (e.g., corticosteroids, vitamin D analogues, retinoids, calcineurin inhibitors, salicylic acid, coal tar, anthralin), light (e.g., sunlight, UVB broadband, UVB narrowband, psoralen plus ultraviolet A (PUVA), excimer laser) or oral or injection medication (e.g., steroids, retinoids, methotrexate, cyclosporine, etanercept (Enbrel), infliximab (Remicade), adalimumab (Humira), ustekinumab (Stelara), secukinumab (Cosentyx), ixekizumab (Taltz), thioguanine (Tabloid), hydroxyurea (Droxia, Hydrea), Apremilast (Otezla)), and may be used in combination with or directly incorporated into the pharmaceutical composition of the present disclosure.

The dosage of the pharmaceutical composition suitable for use according to the present closure can be determined by those skilled in the art on the basis of the disclosure herein. The medicament will contain an effective dosage (depending upon the route of administration and pharmacokinetics of the active agent) of suitable pharmaceutical carriers and excipients suitable for the topical route of administration of the formulation.

In one embodiment, a dosage regimen is repeated, i.e., once, twice, three times or more; for example, repeated for the remaining lifespan of a subject in need. In another embodiment, patients are treated with a dosage regimen of 14 days treatment with a pharmaceutical composition according to the present disclosure. In still another embodiment, patients are treated with a dosage regimen of twice or three times a day for 2 weeks, 3 weeks, 4 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, or 12 months, with a pharmaceutical composition according to the present disclosure.

The present disclosure provides pharmaceutical compositions and methods for treatment of psoriasis. While the pathogenesis of psoriasis has not yet been fully elucidated, significant evidence indicates that epidermal changes occur as a secondary response to cellular immunity infiltrating the skin. Psoriasis is characterized by discrete areas of skin inflammation with redness, thickening, intense scaling, and in some cases, itching. The disease has significant impact on the quality of life of affected individuals, both physically and psychologically. In one embodiment, the treatment is directed at reducing the severity and extent of the psoriatic plaques and the related symptoms. The primary measurement of treatment success used by the U.S. Food and Drug Administration in evaluating products for the treatment of psoriasis is significant overall improvement in psoriasis severity based on Investigators Global Assessment. The following examples are offered to illustrate, but not limit, the claimed invention.

### EXAMPLES

### Example 1 Preparation of Hydrogel of the Present Disclosure

The pharmaceutical composition may be provided as a hydrogel. The embodiments of the formulations of the hydrogels are listed as follows.

TABLE 1

| | | Immunomodulatory | | Ultrapure | |
|---|---|---|---|---|---|
| No. | Gel-forming agent | protein | | water | pH |
| A | 0.8% (w/w) ammonium | 0.005% | (w/w) | Balance | ~6.5 |
| | acryloyldimethyltaurate/ | | | | |
| | VP copolymer | | | | |
| B | 2% (w/w) aloe vera | 0.05% | (w/w) | Balance | ~6 |
| C | 0.1% (w/w) | 0.0001% | (w/w) | Balance | ~6.5 |
| | methylcellulose | | | | |
| D | 0.5% (w/w) xanthan | 0.001% | (w/w) | Balance | ~7 |
| | gum | | | | |
| E | 1% (w/w) sodium | 0.01% | (w/w) | Balance | ~5.5 |
| | alginate | | | | |

The immunomodulatory proteins with SEQ ID NO: 4 were added to ultrapure water and then mixed well. Subsequently, the gel-forming agent was added to the resulting mixture by continuous stirring until the gels were formed. The resulting gels were incubated in a refrigerator at 4° C. for at least 16 hours. The resulting gels were placed in an appropriate container for storage.

### Example 2 Preparation of Capsule of the Present Disclosure

The pharmaceutical composition may be provided as a capsule. Each capsule comprises 350 μg of the immunomodulatory proteins with SEQ ID NOs: 3 or 4.

### Example 3 Treatment of Psoriasis-Patient 1

An 80-year-old female patient 1 suffered from psoriasis on both legs for 60 years since the age of 20. The patient 1 tried many conventional prescriptions, but the condition was still barely controlled.

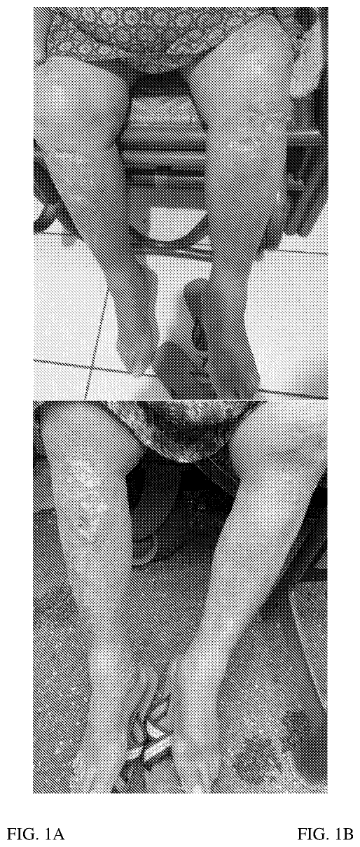

After applying the hydrogel in Example 1 twice a day for 2 months to the left leg, the symptoms of psoriasis on the left leg were eased (FIG. 1 (A) to FIG. 1 (B)).

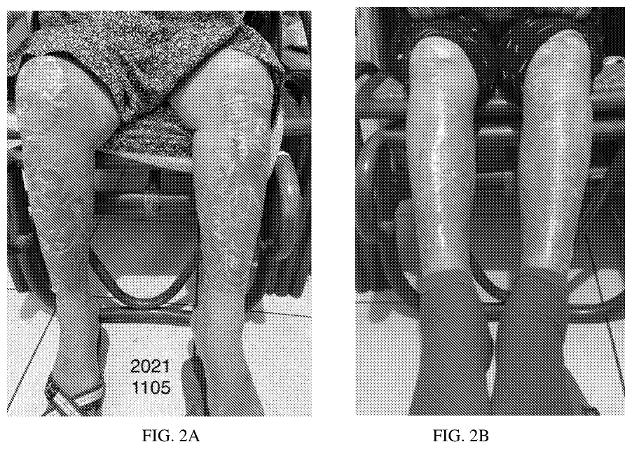

After applying three capsules containing SEQ ID NO: 4 in Example 2 a day for 4 months, the symptoms of psoriasis on the left leg were eased (FIG. 2 (A) to FIG. 2 (B)).

### Example 4 Treatment of Psoriasis-Patient 2

A 53-year-old female patient 2 suffered from psoriasis on both legs for 33 years since the age of 20. The patient 2 tried many conventional prescriptions, but the condition was still barely controlled.

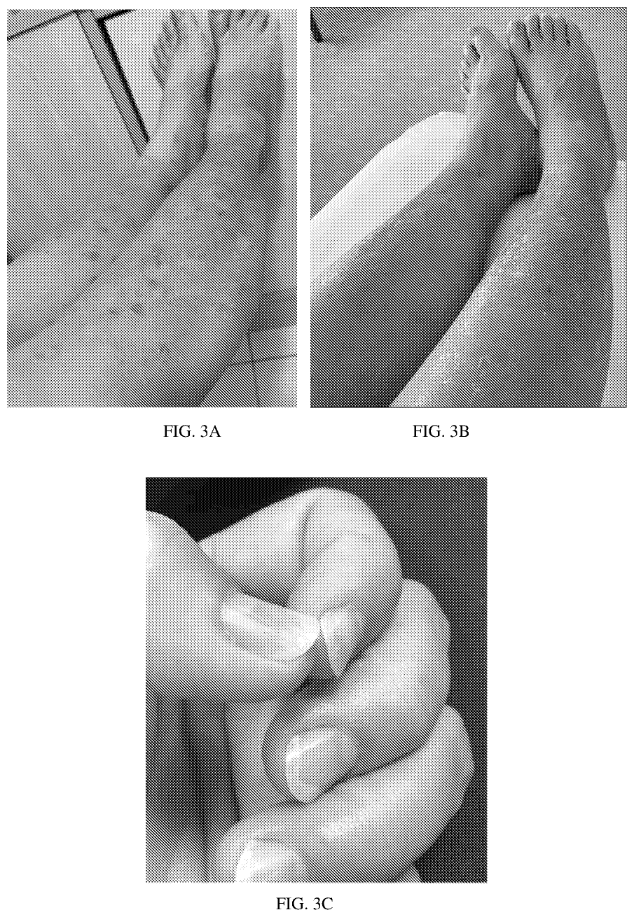

After applying three capsules containing SEQ ID NO: 3 in Example 2 a day for 3 weeks, the symptoms of psoriasis on the skin were eased on week 3 (FIG. 3 (A)), and significantly improved on week 12 (FIG. 3 (B)). Furthermore, the symptoms of psoriasis on the nails were significantly improved on week 12 (FIG. 3 (C)).

### Example 5 Treatment of Familial Psoriasis-Patient 3

A 17-year-old male patient 3 suffered from familial psoriasis, and the psoriatic lesions appeared on the skin.

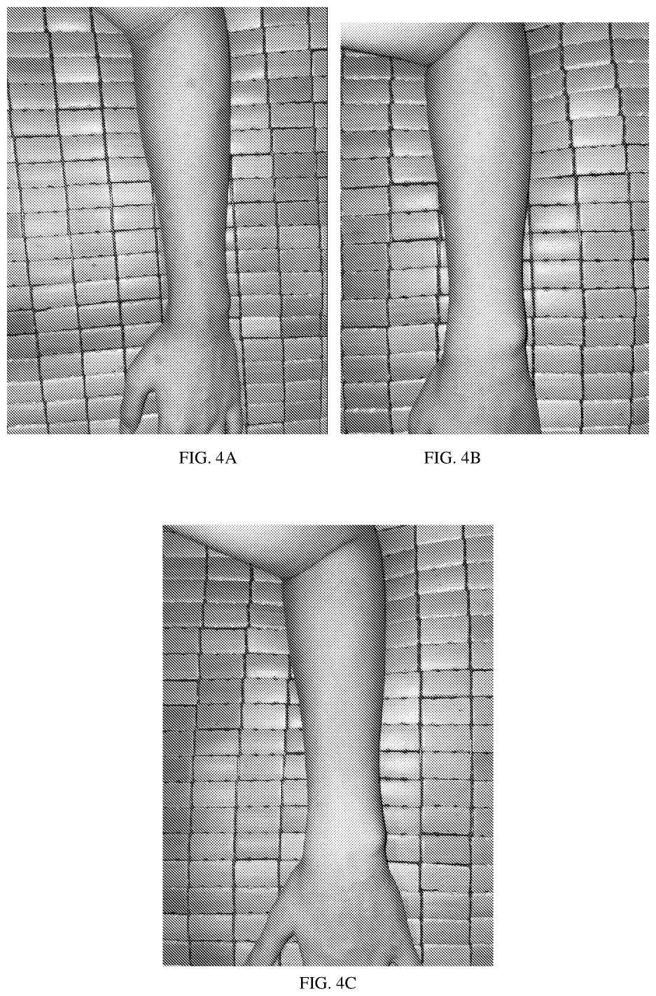

After applying three capsules containing SEQ ID NO: 4 in Example 2 a day for 3 weeks, the symptoms of psoriasis on the skin were significantly eased on week 2 (FIG. 4 (A) to FIG. 4(B)), and on week 4 (FIG. 4 (C)). Furthermore, no recurrence of symptoms for 7 months.

What is claimed is:

1. A method for treating or alleviating psoriasis, and/or promoting or accelerating the healing of psoriasis, comprising administering a pharmaceutical composition to a subject in need thereof, wherein the pharmaceutical composition comprises an effective amount of an immunomodulatory protein derived from Ganoderma, or a recombinant or fragment thereof.

2. The method of claim 1, wherein the immunomodulatory protein comprises an amino acid sequence of SEQ ID NO: 3 or 4.

3. The method of claim 1, wherein the fragment of immunomodulatory protein comprises an amino acid sequence selected from the group consisting of SEQ ID NOs: 1 to 2.

4. The method of claim 1, wherein the pharmaceutical composition further comprises a gel-forming agent.

5. The method of claim 4, wherein the gel-forming agent is in an amount from about 0.1% (w/w) to about 2% (w/w) and the immunomodulatory protein is in an amount from about 0.0001% (w/w) to about 0.05% (w/w) in the pharmaceutical composition.

6. The method of claim 4, wherein the gel-forming agent is polyethylene glycol (PEG)-diacrylate, PEG-acrylate, PEG-thiol, PEG-azide, PEG-alkyne, chitosan, hyaluronic acid, collagen, fibrin, acacia, alginic acid, natto gum, aloe vera, bentonite, carbomers, carboxymethyl cellulose, ethylcellulose, gelatin, elastin, hydroxypolyamide, hydroxyethyl cellulose, hydroxypropyl cellulose, hydroxypropyl methylcellulose, magnesium aluminum silicate, methylcellulose, poloxamers, polyvinyl alcohol, sodium alginate, tragacanth, xanthan gum, gelatin, carboxyvinyl polymers, starch, water-swellable hydrocolloids, carragenans, hyaluronates, agarose, alginates, acrylates or ammonium acryloyldimethyltaurate/vinyl pyrrolidone (VP) copolymer or a combination thereof.

7. The method of claim 4, wherein the gel-forming agent is xanthan gum, methylcellulose, natto gum, aloe vera, or ammonium acryloyldimethyltaurate/VP copolymer.

8. The method of claim 4, wherein the pharmaceutical composition has a pH value ranging from 5.5 to 7.5.

9. The method of claim 4, wherein the pharmaceutical composition has a viscosity ranging from about 0.05 Pa·s to about 200 Pa·s.

10. The method of claim 4, wherein the amount of the immunomodulatory protein ranges from about 0.0001% (w/w) to about 0.03% (w/w).

11. The method of claim 4, wherein the amount of the gel-forming agent ranges from about 0.5 (w/w) to about 1.2% (w/w).

12. The method of claim 4, wherein the pharmaceutical composition is topically administered on an area of psoriasis.

13. The method of claim 12, wherein the effective amount of the immunomodulatory protein ranges from about 1 mcg/cm² to about 100 mcg/cm² of the area of psoriasis.

14. The method of claim 1, wherein the pharmaceutical composition is orally administered to the subject.

15. The method of claim 14, wherein the effective amount of the immunomodulatory protein ranges from about 0.01 mg/kg to about 5 mg/kg.

16. The method of claim 14, wherein the effective amount of the immunomodulatory protein ranges from about 0.1 mg/kg to about 3 mg/kg.

17. The method of claim 1, wherein the method further comprises administering one or more therapeutic agents to the subject.

18. The method of claim 1, wherein the psoriasis is plaque psoriasis, pustular psoriasis, inverse psoriasis, napkin psoriasis, guttate psoriasis, oral psoriasis, erythrodermic psoriasis, seborrheic-like psoriasis, nail psoriasis, or psoriatic arthritis.

19. The method of claim 1, wherein the psoriasis is familial psoriasis.


SEQUENCE LISTING (4 sequences)

SEQ ID NO: 1 - protein, 6 aa, Ganoderma microsporum
LAWNVK

SEQ ID NO: 2 - protein, 10 aa, Ganoderma microsporum
DLGVRPSYAV

SEQ ID NO: 3 - protein, 111 aa, Ganoderma microsporum
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 4 - protein, 140 aa, Artificial Sequence
  recombinant protein
EAEAEFMSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWNYLEQKLISEEDLNSAVDHHHHHH